(19) United States
(12) Patent Application Publication
(10) Pub. No.: US 2025/0050013 A1
(43) Pub. Date: Feb. 13, 2025

(54) SYSTEMS, METHODS, AND COMPONENTS THEREOF FOR IMPROVING FLUID FLOW DURING MEDICATION DELIVERY PROCEDURES

(71) Applicant: Damea Alexander, Marysville, OH (US)

(72) Inventor: Damea Alexander, Marysville, OH (US)

(21) Appl. No.: 18/927,106

(22) Filed: Oct. 25, 2024

Related U.S. Application Data
(63) Continuation-in-part of application No. 17/980,173, filed on Nov. 3, 2022.
Application No. 17/980,173 is a division of application No. 17/093,778, filed on Nov. 10, 2020, now Pat. No. 12,023,466.

(51) Int. Cl. A61M 5/162; A61J 1/20; A61M 5/14; A61M 5/165; A61M 5/168; A61M 39/00; A61M 39/10; A61M 39/28

(52) U.S. Cl. CPC A61M 5/162; A61J 1/2006; A61J 1/2068; A61M 5/165; A61M 5/16881; A61M 39/10; A61M 39/28; A61M 5/1411; A61M 2005/1623; A61M 2039/0081

(57) ABSTRACT

A snorkel for improving fluid flow during medication delivery procedures is disclosed along with related delivery systems and methods. The snorkel includes a fluid passageway forming an interior passageway for ambient air. A fluid control device is located at the fluid passageway and is moveable between a closed position and an opened position to control passage of the ambient air at the interior passageway. The fluid passageway is sized and shaped to permit extension of a first end of the fluid passageway through a seal of a container and above a fluid line of a substance within the container while the container is oriented for infusion or pooling, the substance remains undrained, and a second end of the fluid passageway as well as the fluid control device remains outside of, and spaced apart from, the container.

### CROSS-REFERENCE TO RELATED APPLICATIONS

This application is a continuation-in-part of U.S. application Ser. No. 17/980,173 filed Nov. 3, 2022, which is a divisional of U.S. application Ser. No. 17/093,778 filed Nov. 10, 2020, the disclosures of each of which are hereby incorporated by reference as if fully restated herein.

### TECHNICAL FIELD

Exemplary embodiments relate generally to systems, methods, and components thereof, such as a snorkel, for improving fluid flow during medication delivery procedures, such as infusions or pooling.

### BACKGROUND AND SUMMARY OF THE INVENTION

Medication often needs to be delivered from one location to another for a variety of reasons. Examples of such procedures include, but are not limited to, infusions and pooling. Infusions or pooling are performed for a variety of medical reasons. Infusions may be performed as part of a treatment regimen for certain acute or chronic disease. For example, without limitation, certain autoimmune disorders may require periodic infusions of immunoglobulin, sometimes for the remainder of the patient's life. These medications after often expensive and may be difficult to obtain. Certain such infusions treatments may account for a significant amount of insurance expenditures. Traditionally, delivery systems are used which include a spike. The spike is inserted into a container. A pathway is provided through the spike for draining fluid from the container. Tubing fluidly connects the spike to an intravenous device inserted into a patient's vascular system such that the medication may be delivered to the patient.

In another similar procedure, pooling, medication is transferred from one container to another. Similar steps are generally followed, but the tubing is connected to a second container to which the medication is transferred. For example, medication may be transferred from a rigid container, such as a bottle, to a flexible one, such as a bag. This may be performed for preferences, to dilute medication, to combine medications or otherwise create solutions or other compounds, and/or to share the medication among multiple patients. Typically, the medications or other substances being infused or pooled are provided in liquid form. Such medications may likewise be expensive and are often difficult to obtain.

Traditional spikes may be vented or non-vented. Generally, vented spikes are not needed where the container is collapsible, such as with bags. However, vented spikes are generally required where the container is rigid, such as with glass or rigid plastic containers. Where vented spikes are used, the vent may be opened to permit outside air to enter the container, thereby allowing the substance in the container to drain for administration to the patient or transfer to another container. A valve may be provided at the vent to permit air to enter the container while preventing the substance from exiting. An example of a vented spike is provided in U.S. Pat. No. 4,857,068. The air ingested through the vent may rise through the substance in the container, generally causing the substance in the container to foam. The foamed substance cannot be readily delivered to the patient, and thus generally remains in the container after administration or transfer is ceased. This generally results in significant amounts of undelivered substance left remaining in the container which is never delivered to the patient. Therefore, what is needed are systems and methods for improving fluid flow during infusions or pooling.

Systems and methods are provided which improve fluid flow during medication delivery procedures, such as but not limited to, infusions and pooling. Such systems and methods may be designed to provide smooth draining from a container during such procedures, thereby reducing or eliminating foaming and increasing the amount of substances administered to the patient, transferred, or otherwise delivered. In exemplary embodiments, the spike may comprise a channel configured to accommodate a snorkel in a sliding manner. The snorkel may be inserted into the channel of the spike. A first, open end of the snorkel may be elevated above a fluid level of the substance within the container to permit air to enter the container during infusion, pooling, or the like to permit draining of the substance. Because the air may enter above the substance, without passing directly through it, foaming may be reduced or eliminated.

A protective cover may be provided from an entrance of the channel to a second, open end of the snorkel. The protective sleeve may be collapsible so that the snorkel may be selectively advanced into the container and retracted therefrom. This may permit the snorkel to be inserted and removed from the container while maintaining a sterile environment. In this way, the snorkel may be reused in multiple containers. For example, without limitation, this may permit multiple containers to be used during an infusion, pooling, or other procedure. For example, without limitation, this may permit utilization with different size containers. A valve, port, or the like may be provided on an end of, and/or along, the snorkel. In this way, the substance in the container may be prevented from exiting through the snorkel while being inserted through the substance.

The snorkel may snugly fit within the channel such that the snorkel may be temporarily secured at locations along the channel. Alternatively, or additionally, a clamp or other device may be used to secure the snorkel relative to the channel.

Alternatively, or additionally, the snorkel may be inserted through the seal of the container adjacent to the spike. The seal may be of sufficient size to accommodate both a portion of the spike and the snorkel. In such embodiments, the spike may include the pathway for draining the substance. The channel for the snorkel may not be required, and traditional vented or unvented spikes may be used. The seal and/or the snorkel may comprise one or more materials having sufficient frictional properties to permit the snorkel to be temporarily secured at various positioned relative to said seal. For example, without limitation, the seal may comprise a rubberized material and the snorkel may comprise a polymer, metal, or the like. A fluid control device may be provided on a second end of the snorkel. A first end of the snorkel may be blunted, though such is not required.

Alternatively, or additionally, an airflow control device may be provided at the container. The airflow control device may comprise a valve, such as but not limited to a one-way valve, port, or the like. The airflow control device may be positioned above the fluid line of the substance when the container is positioned for administration, transfer, or other dispensation. For example, without limitation, the airflow control device may be located on or near the bottom surface of the container.

In exemplary embodiments, without limitation, the snorkel may include a handle for a user to grip and manipulate, such as to move the snorkel relative to the container for the medication. The handle may include a removable cap that acts to at least partially, if not fully, block or seal a fluid passageway of the snorkel, thereby acting as a fluid control device. The cap may be fully removable, or may remain attached in part to the handle, such as to allow hinging movement which removes the cap from a first end of the handle. A filter may be located below the cap, such as to filter air entering an interior passageway of the fluid passageway when in use, such as to prevent or inhibit debris or other objects from entering the container. The fluid passageway may extend through, or at least partially within, the handle. A first end of fluid passageway may be inserted into the container and raised above the fluid line to allow smooth draining. A portion of the fluid passageway may extend within at least a cover extending from, or forming part of, a second end of the handle.

The fluid passageway may include one or more segment(s). The fluid passageway may include at least one non-linear segment (e.g., curved), and at least one linear segment. A longitudinal axis of the handle may extend at a non-zero, non-180-degree angle (e.g., between 10-80 degrees, without limitation) relative to at least a longest one of the at least one linear segment, thereby providing a comfortable handing orientation of the snorkel.

The portion of the fluid passageway within the handle may form a funnel shape, such as to accommodate the filter and/or cap, though other shapes may be utilized.

Further features and advantages of the systems and methods disclosed herein, as well as the structure and operation of various aspects of the present disclosure, are described in detail below with reference to the accompanying figures.

### BRIEF DESCRIPTION OF THE DRAWINGS

In addition to the features mentioned above, other aspects of the present invention will be readily apparent from the following descriptions of the drawings and exemplary embodiments, wherein like reference numerals across the several views refer to identical or equivalent features, and wherein:

FIG. 1 is a plan view of a traditional delivery system;

FIG. 2A is a front view of an exemplary container illustrating an exemplary substance after administration or transfer using the traditional delivery system of FIG. 1;

FIG. 2B is a bottom view of the container of FIG. 2A after the foamed substance is allowed to condense;

FIG. 3A is a side sectional view of an exemplary delivery system with improved fluid flow in accordance with the present invention;

FIG. 3B is a side sectional view of the delivery system of FIG. 3A with a snorkel partially inserted, such as for use with a relatively small container;

FIG. 3C is a side sectional view of the delivery system of FIG. 3A with the snorkel fully inserted, such as for use with a relatively large container;

FIG. 4 is a side sectional view of an exemplary spike of the delivery system of FIGS. 3A-3C, shown in isolation from other components of the delivery system;

FIG. 5 is a side view of the snorkel of the delivery system of FIGS. 3A-3C, shown in isolation from other components of the delivery system;

FIG. 6A is a side sectional view of another exemplary delivery system with improved fluid flow in accordance with the present invention;

FIG. 6B is a side view of the snorkel of the delivery system of FIGS. 6A, shown in isolation from other components of the delivery system;

FIG. 7 is a flow chart for an exemplary method for using the delivery system of FIG. 3A-6B in accordance with the present invention;

FIG. 8A is a side sectional view of another exemplary delivery system with improved fluid flow in accordance with the present invention, also illustrating detail A;

FIG. 8B is a detailed side sectional view of Detail A of FIG. 8A;

FIG. 8C is a detailed side sectional similar to FIG. 8B with an exemplary needle inserted; and

FIG. 9 is a flow chart for an exemplary method of using the delivery system of FIGS. 8A-8C in accordance with the present invention;

FIG. 10A is a top perspective view of another exemplary embodiment of the snorkel in accordance with the present invention, such as for use with the delivery systems and/or methods of FIGS. 1-9;

FIG. 10B is a bottom perspective view of the snorkel of FIG. 10A;

FIG. 10C is top perspective view of the snorkel of FIGS. 10A-10B with a cap not illustrated;

FIG. 10D is a top view of the snorkel of FIGS. 10A-10C with a filter not illustrated; and

FIG. 10E is a side perspective view of fluid passageway segment(s) of the snorkel of FIGS. 10A-10E illustrated in isolation.

### DETAILED DESCRIPTION OF EXEMPLARY EMBODIMENT(S)

Various embodiments of the present invention will now be described in detail with reference to the accompanying drawings. In the following description, specific details such as detailed configuration and components are merely provided to assist the overall understanding of these embodiments of the present invention. Therefore, it should be apparent to those skilled in the art that various changes and modifications of the embodiments described herein can be made without departing from the scope and spirit of the present invention. In addition, descriptions of well-known functions and constructions are omitted for clarity and conciseness.

Embodiments of the invention are described herein with reference to illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

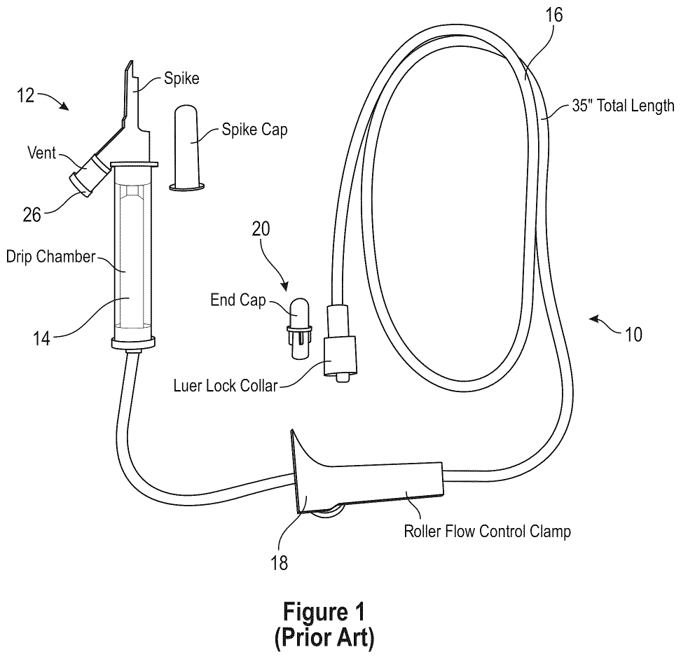

FIG. 1 illustrates a traditional delivery system 10. The delivery system 10 may be used for various medication delivery procedures, such as but not limited to, infusions and pooling. The traditional delivery system 10 may include a spike 12 connected to a drip chamber 14. The drip chamber 14 may be connected to a first end of tubing 16. A flow control device 18 may be provided along the tubing 16. One or more adapters or devices 20 may be connected to a second end of the tubing 16 for providing, or connecting to devices which provide, access to a patient's vascular system, such as but not limited to needles, ports, or the like. The spike 12 may comprise a vent 26, or may be ventless.

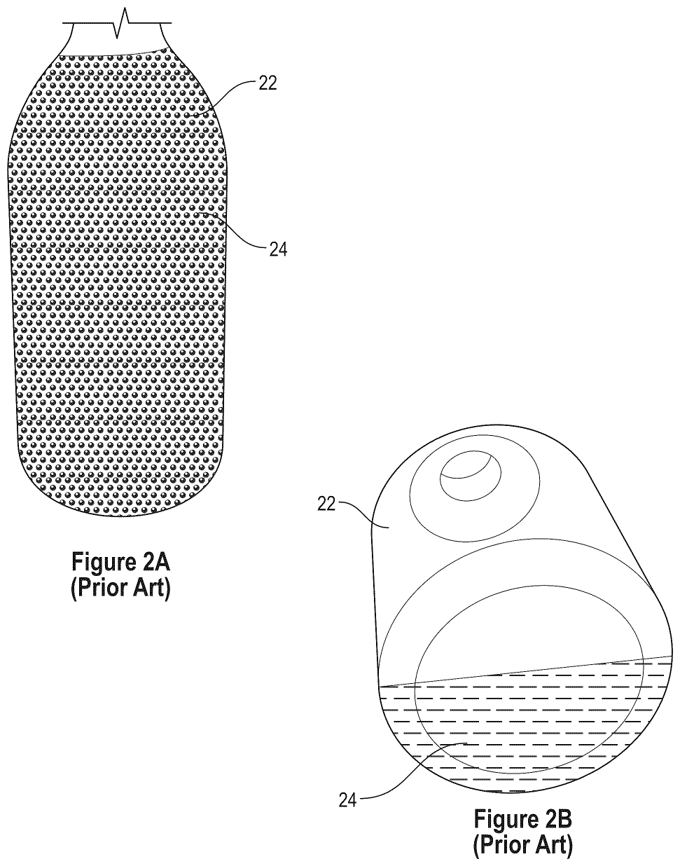

FIG. 2A illustrates an exemplary container 22 with a foamed substance 24 after administration or transfer using the traditional delivery system 10. The spike 12 may be used to puncture a seal provided on the container 22. The container 22 may be inverted to allow the substance 24 to be administered or transferred, at least in part, by gravitational forces. The vent 26 on the spike 12, if provided, may be left closed when the container 22 is punctured by the spike 12. The drip chamber 14 may be squeezed to begin the flow of the liquid substance 24 while the vent 26 remains closed. The vent 26 may subsequently be opened to permit air to enter the container 22 so that the liquid substance 24 may be slowly drained from the contained 22 for administration to the patient, transfer to another container, or other delivery. The vent 26 may incorporate a valve or cap for controlling air flow. For example, the vent 26 may be configured to allow airflow into the container 22 while preventing the substance 24 from existing the container through the vent 26. Because the vent 26 is located below a fluid line of the substance 24, the air entering the container 22 may be forced to bubble up through the liquid substance 24, which may result in the illustrated foaming shown in FIG. 2A. The spike 12 may not be configured to accept the foamed substance 24, and thus the foamed substance 24 is typically left un-administered to the patient, un-transferred to another container 22, or otherwise undelivered to a desired location.

FIG. 2B illustrates the foamed substance 24 being allowed to condense back down to a liquid. FIG. 2B provides an example of the amount of substance 24 which may be left in the container 22, un-administered to the patient, un-transferred, or otherwise un-removed. It may take a significant amount of time, up to several hours for example, for the foamed substance 24 to re-condense. As such, this leftover substance 24 is typically discarded.

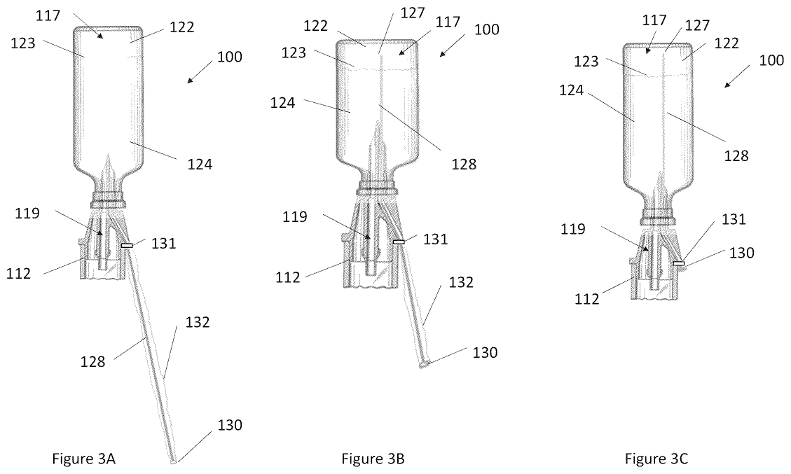

FIG. 3A through FIG. 3C illustrate an exemplary medication delivery system with improved fluid flow 100 (hereinafter also the “system 100”). Similar items may be numbered similarly but increased by 100 (e.g., 12 to 112). Any type or kind of container 122 may be utilized.

The system 100 may be used to delivery any type or kind of substance 124, which is typically provided as a liquid. The substance 124 may comprise any number and type of ingredients. The substance 124 may be provided as a solution, such as a colloid solution, compound, or the like. Typically, at least one medication and at least one inactive carrying fluid are provided in the substance 124, though such is not required. Any type or kind of inactive ingredient, or combination of inactive ingredients, may be utilized. Any type or kind of medication, or combination of medications, may be utilized. The medications may comprise one or more naturally occurring substances, synthesized substances, combinations thereof, or the like. For example, at least one of the medications may comprise one or more proteins or immunoglobulin, such as but not limited to, albumin. These substances may be expensive and/or difficult to obtain. In the example of Immunoglobulin, which is provided without limitation, a single container 122 which is typically administered to a single patient, may be derived from plasma donations from multiple individuals. With the scarcity of available plasma donations, it is readily apparent why discarding even relatively small amounts of remaining substance 124 is undesirable.

In exemplary embodiments, the container 122 may comprise a bottle or otherwise rigid or semi-rigid container. The container 122 may comprise glass, plastic, or the like. The container 122 may comprise a puncturable seal at an otherwise open end for receiving a spike 112. In other exemplary embodiments, the container 122 may be flexible, such as but not limited to, where the container 122 comprise a bag. Even in the case of such flexible containers, certain amounts of substance 124 may remain in the container 122, such as within creases or air pockets created during administration, transfer, or other delivery. Thus, use of the delivery system 100 may also provide advantages when used with such flexible containers 122.

The spike 112 may comprise one or more pathways 119 for draining the substance 124 from the container 122.

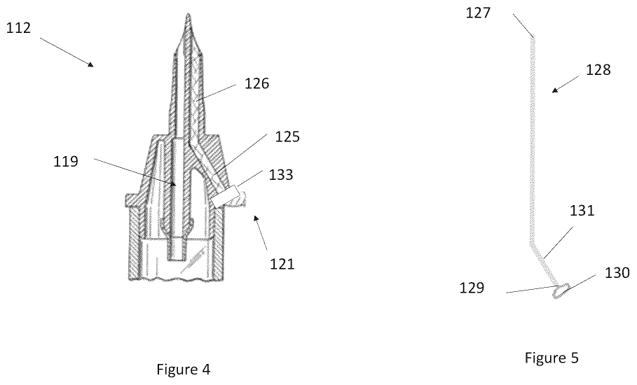

As illustrated particularly in FIG. 4, the spike 112 may comprise a channel 126. The channel 126 may provide a pathway through the spike 112 between the container 122 and the ambient environment when said spike 112 is inserted into the container 122, such as by puncturing the seal.

The channel 126 may be configured to accommodate a snorkel 128. The channel 126 may comprise one or more non-linear section 125, such as but not limited to angular sections, curves, sinuous passages, or the like, though such is not required. For example, in other exemplary embodiments, the channel 126 may extend in an entirely straight line through the spike 112. The snorkel 128 may comprise one or more sufficiently flexible materials to negotiate the non-linear section 125 of the channel 126. Alternatively, or additionally, the snorkel 128 may comprise one or more sufficiently rigid material to remain stiff and upright when inserted into the container 122. The snorkel 128 may comprise a polymer, metal, combinations thereof, or the like. Some or all of the snorkel 128 may be hollow to facilitate the flow of fluids, such as but not limited to air, through the snorkel 128.

The snorkel 128 may be configured for sliding movement within the channel 126. The snorkel 128 may be moveable between a retracted position (see e.g., FIG. 3A), to an extended position (see e.g., FIG. 3C), or be secured at a position between such extended and retracted positions (see e.g., FIG. 3B). The snorkel 128 and/or the spike 112, or components thereof, may be configured to permit the snorkel 128 to be temporarily secured at the retracted position, the extended position, and/or positions therebetween. In exemplary embodiments, the fit between the snorkel 128 and the channel 126 may be sufficiently snug to permit a press or friction fit that secure the position of the snorkel 128 relative to the spike 112. For example, without limitation, a force greater than a predetermined amount may be required to move the snorkel 128 through the channel 126.

In other exemplary embodiments, a securing device 133 may be provided at, or connected to, the spike 112. The securing device 133 may be configured to secure the position of the snorkel 128 relative to the channel 126. The securing device 133 may comprise a strap and/or clamp for compressing the snorkel 128, such as but not limited to a C-clamp, hose clamp, tubing clamp, utility clamp, some combination thereof, or the like. Any type of kind of securing device 133 may be utilized. In exemplary embodiments, the securing device 133 may be positioned at or near the entrance 121 of the channel 126, though any location may be utilized. For example, without limitation, the securing device 133 may be positioned along the channel 126. The securing device 133 may be normally secured, normally unsecured, or the like.

As illustrated particularly in FIG. 5, the snorkel 128 may comprise a hollow member. The snorkel 128 may comprise a first end 127 and a second end 129 which may be opposite the first end 127. One or both of the first and second ends 127, 129 may be open, though in other exemplary embodiments, openings may be provided at or near the first or second end 127, 129.

In the retracted position, the first end 127 of the snorkel 128 may reside at least partially within the channel 126. In the extended position, the first end 127 of the snorkel 128 may extend within the container 122. In exemplary embodiments, the snorkel 128 may be of sufficient length to extend through the entire container 122 and the spike 112 when fully inserted (e.g., when in the extended position). At a minimum, the snorkel 128 may be of sufficient length to permit the first end 127 to be elevated above a fluid level of the substance 124 in the container 122 when the snorkel 128 is in the extended position.

The snorkel 128 may comprise a fluid control device 130 at the second end 129 thereof. The fluid control device 130 may be configured to permit air to enter the snorkel 128 (and thus the container 122) while preventing the substance 124 from exiting the container 122 through the snorkel 128. Alternatively, or additional, the fluid control device 130 may be configured to prevent certain outside contaminants (e.g., dust, pollen, germs, smoke, combinations thereof, or the like) from entering the container 122 while air is ingested. The fluid control device 130 may comprise a filter, cap, valve (e.g., one- or two-way valve, check value), combinations thereof, or the like. In other exemplary embodiments, a fluid control device 130 may not be required and a finger, other appendage, or other blocking member may be placed over the second end 129, which may be otherwise open, to control fluid flow. Any number and type of fluid control devices 130 may be utilized, such as but not limited to, along the snorkel 128, at the first end 127 of the snorkel 128, combinations thereof, or the like. The first end 127 may be otherwise be open.

The delivery system 100 may comprise a protective cover 132. The protective cover 132 may include one or more adjustable length members extending between the channel 126 and the second end 129 of the snorkel 128. The protective cover 132 may be connected to the spike 112 at a first end of the cover 132. The protective cover 132 may be connected to the fluid control device 130 at a second end of the cover 132. The protective cover 132 may comprise a sheath, sleeve, combinations thereof, or the like which is capable of collapsing. The protective cover 132 may, alternatively or additionally, comprise one or more telescoping member, compressible tubing, combinations thereof, or the like. The protective cover 132 may be configured to cover a portion of the snorkel 128 extending from the channel 126. The protective cover 132 may be configured for collapsing or otherwise reducing to a size which permits the snorkel 128 to be fully extended within the channel 126, and the container 122. The protective cover 132 may be configured for expansion or otherwise increasing to a size which permits the snorkel 128 to be fully retracted from the channel 126, and thus the container 122. The protective cover 132 may permit the snorkel 128 to remain in a sterile environment despite retraction and extension. This may permit the spike 112 and/or the snorkel 128 to be reused. Alternatively, or additionally, this may permit utilization of the snorkel 128 with different size containers 122. For example, the protective cover 132 may provide a sterile environment even where a portion of the snorkel 128 is extended form the channel 126, which may be the case when relatively short containers 122 are used. For example, FIGS. 3A and 3C illustrate use with a relatively large container 122, and FIG. 3B illustrates use with a relatively small container 122.

The channel 126 may comprise one or more non-linear sections 125. The snorkel 128 may comprise sufficiently flexible materials to permit the snorkel 128 to negotiate the non-linear sections 125 of the channel 126. The snorkel 128 may comprise materials with sufficient durability and/or memory that the snorkel 128 resumes a linear orientation upon passage beyond the non-linear sections 125.

Alternatively, or additionally, the snorkel 128 may comprise one or more non-linear segments 131. The non-linear segments 131 may be configured to accommodate some or all of the non-linear sections 125 of the channel 126. For example, the snorkel 128 may be shaped to match the channel 126 when the snorkel 128 is fully inserted within the channel 126, such as when the snorkel 128 is the extended position. The non-linear nature of the sections 125 and/or segments 131 may provide the necessary fiction to temporarily secure the snorkel 128 relative to the channel 126. The snorkel 128 may comprise a sufficiently durable, memory material that normally takes a shape which include the non-linear segments 131, such as but not limited to one or more polymers.

The first end 127 of the snorkel 128 may be sharpened, blunted, or the like.

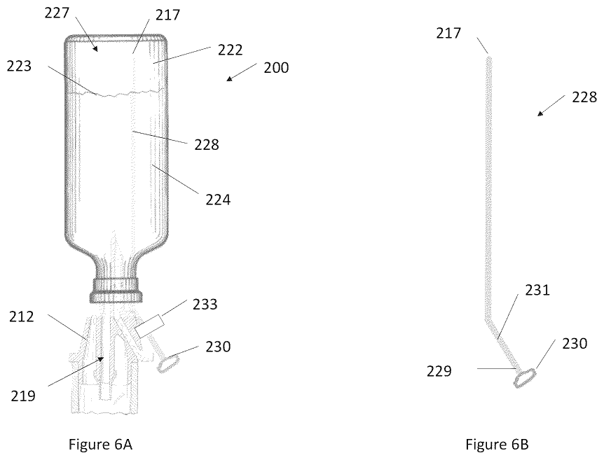

FIG. 6A and FIG. 6B illustrate another exemplary delivery system with improved fluid flow 200 (hereinafter also the “system 200”). Similar items may be numbered similarly but increased by multiples of 100 (e.g., 112 to 212). A snorkel 228 may be inserted adjacent to a spike 212. In exemplary embodiments, a container 222 may comprise a puncturable seal located on an otherwise open end thereof. The seal may be of sufficient size to accommodate at least a portion of the spike 212 and the snorkel 228. The seal may comprise a foil, rubberized material, combinations thereof, or the like. A first end 217 of the snorkel 228 in such embodiments may be blunted, though a sharpened or other type of end may be utilized. The first end 217 may be open or the opening may be located a distance from the termination of the first end 217. The snorkel 228 may be punctured through said seal adjacent to at least the portion of the said spike 212 punctured through the seal.

The snorkel 228 may comprise one or more non-linear segments 231. The non-linear segments 231 may permit a second end 229 of the snorkel 228 to extend a distance from an outer surface of the spike 212 to facilitate access to said second end 229 when said snorkel 228 is inserted into said container 222. Any shape and number of non-linear segments 231 may be utilized. The second end 229 of the snorkel 228 may be open or the opening may be located a distance from the termination of the second end 229.

One or more fluid control devices 230 may be provided at the second end 229 of the snorkel 228, along the snorkel 228, at the first end 217, combinations thereof, or the like. The fluid control devices 230 may comprise a cap, valve, filter, combinations thereof, or the like.

The spike 212 may be vented or unvented. The spike 212 may comprise one or more pathways 219 for draining a substance 224 from said container 222. Stated another way, the spike 212 may not require a separate channel, such as channel 126, for the snorkel 228 as the snorkel 228 may be inserted into the container 222 adjacent to the spike 212. Traditional spikes (vented or non-vented) may be used with the delivery system 200, though such is not required.

The snorkel 228 may be moveable between a retracted position where said snorkel 228 is located entirely outside of said container 222, and an extended position whereby said first end 217 of said snorkel 228 extends above a fluid line 223 (actual or anticipated) of a substance 224 in said container 222 and into an air pocket 217 (actual or anticipated). The snorkel 228 and/or the seal of the container 222 may comprise one or more materials having sufficient frictional properties that said snorkel 228 is temporarily securable at said extended position, or a position between said retracted position and said extended position. Alternatively, or additionally, a securement device 233, such as but not limited to a clamp, strap, or the like, may be provided at said spike 212 for securing said snorkel 228.

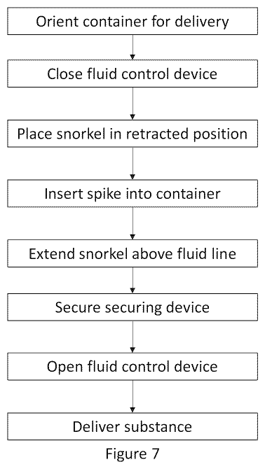

FIG. 7 provides an exemplary flowchart for use of the system 100 and/or 200. The snorkel 128 may be provided at least partially inserted into the channel 126 of the spike 112. In other exemplary embodiments, the snorkel 128 may be provided entirely outside of the channel 126 and may be first inserted into the channel 126. In yet other exemplary embodiments, the snorkel 228 may be provided entirely outside of the spike 212 and inserted adjacent to the spike 212. The protective cover 130 may be attached to the snorkel 128 and/or the channel 126. The spike 112, 212 may be inserted into the container 122, 222. The snorkel 128, 228 may be extended such that a first end 127,227 of the snorkel 128, 228 is raised into an air pocket 117, 217 (actual or anticipated) located above a fluid line 123,223 (actual or anticipated) of the substance 124, 224 in the container 122, 222. Stated another way, the snorkel 128, 228 may be raised into the actual air pocket 117, 217 located above the actual fluid line 123, 223 if the container 122, 222 is already positioned for delivery of the substance 124, 224. Alternatively, the snorkel 128, 228 may be raised into the anticipated air pocket 117, 217 located above the anticipated fluid line 123, 223 if the container 122, 222 is not yet positioned for delivery of the substance 124, 224.

Regardless, the position of the snorkel 128, 228 within the air pocket 117, 217 above the fluid line 123, 223 may permit the introduction of ambient air into the container 122, 222 without the need for the air to bubble directly through the substance 124, 224 once the container 122, 222 is positioned for delivery of the substance 124, 224 and the delivery of the substance 124, 224 has begun, thereby permitting smooth draining of the substance 124, 224 during delivery, which may decrease or eliminate foaming and permit increased or full delivery of the substance 124, 224. Where the fluid control device 130, 230 is utilized, the fluid control device 130, 230 may be closed before the snorkel 128, 228 is raised above the fluid line 123, 223, and opened after the snorkel 128, 228 is raised above the fluid line 123, 223. In other exemplary embodiments, the fluid control device 130, 230 may be configured to automatically prevent the substance 124, 224 from traveling therebeyond, but permit ambient air to enter (e.g., one-way value). The position of the snorkel 128, 228 may be secured by the securing device 133, 233.

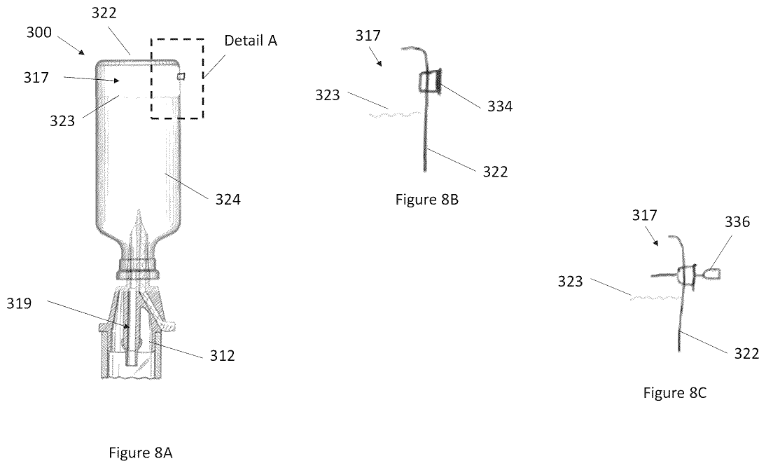

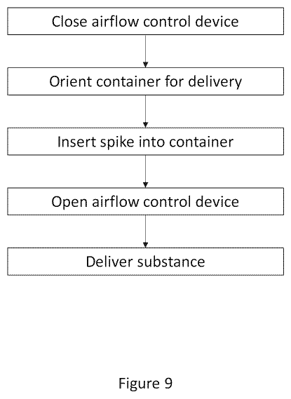

FIG. 8A through FIG. 8C illustrate another exemplary delivery system with improved fluid flow 300 (hereinafter also the “system 300”). FIG. 9 illustrates an exemplary method for using the delivery system 300. Similar items may be numbered similarly but increased by multiples of 100 (e.g., 112 to 212 or 312). The delivery system 300 may comprise an airflow control device 334 provided at the container 322. The airflow control device 334 may be configured to selectively permit ambient air to enter the container 322.

The airflow control device 334 may comprise a port, cap, stopper, puncturable seal, valve (one-way, two-way, check, combinations thereof, or the like), combinations thereof, or the like. In exemplary embodiments, without limitation, the airflow control device 334 may comprise a rubber stopper. The airflow control device 334 may be positioned at a location of the container 322 within an air pocket 317 (actual or anticipated) located above a fluid line 323 (actual or anticipated) when the container 322 is positioned for administration, transfer, or other removal through a pathway 319. For example, without limitation, the airflow control device 334 may be positioned at or adjacent to a bottom surface, or at bottom portion of the container 322 such that when the spike 312 is inserted and the container 322 is oriented for delivery of the substance 324 (e.g., inverted) the airflow control device 334 is located above the fluid line 323. In this way, the air entering the container 322 does not pass through the substance 324, and thus does not create foaming. Any number of airflow control devices 334 may be utilized or the same or different type at any location on the container 322.

In exemplary embodiments, the airflow control device 334 may be configured to be remain sealed until a needle 336 or other device is inserted through the airflow control device 334 to permit air to enter the container 322. The airflow control device 334 may be configured to remain closed until certain user interaction is provided, such as but not limited to, opening a cap, removing a stopper, opening a valve, combinations thereof, or the like. Alternatively, the airflow control device 334 may be configured to remain open until certain user interaction is provided. The needle 336 may comprise a filter configured to prevent particulate of a predetermined size from entering said container 322 even when said airflow control device 334 is opened. In this way, contaminants (e.g., dust, pollen, smoke, germs, combinations thereof, or the like) may be prevented from entering the container 322 while the airflow control device 334 is opened or otherwise fully or partially unsealed.

The delivery system 300 may be used as an alternative to, or in conjunction with, the delivery system 100 and/or the delivery system 200. For example, without limitation, the snorkel 128, 228 and/or spike 112, 212 may be used in combination with the airflow control device 334, though such is not required.

Traditional spikes (vented or non-vented) may be used with the delivery system 300, though such is not required. The delivery system 300 may be used with rigid or semi-rigid containers 322, such as bottle. In other exemplary embodiments, the delivery system 300 may be used with flexible containers 322, such as bags. Even in the case of flexible containers 322, certain amounts of substance 324 may remain in the container 322, such as within creases or air pockets created during administration, transfer, or other removal. Thus, the delivery system 300 may have applicability to such flexible containers 322.

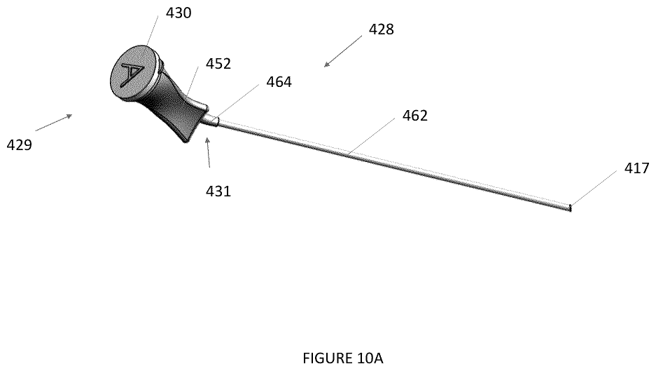

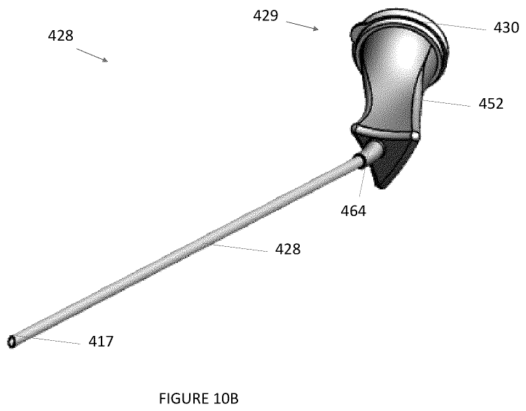

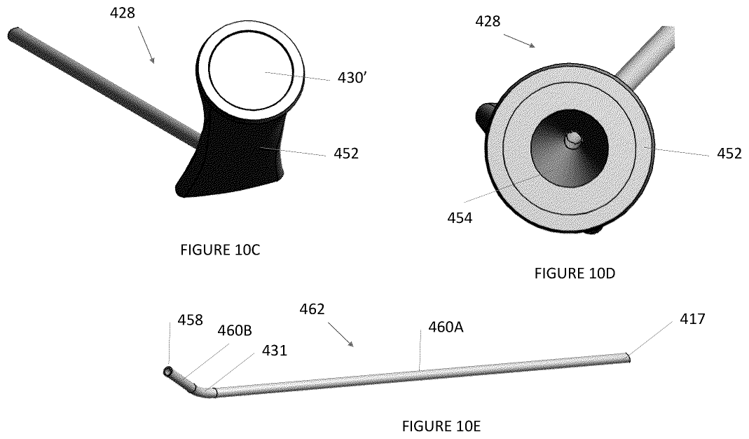

FIG. 10A through FIG. 10E illustrate another exemplary embodiment of the snorkel 428. Similar items may be numbered similarly but increased by increments of 100 (e.g., 128 and/or 228 to 428). The snorkel 428 may be used with some or all components the delivery systems 100, 200, 300 and/or some or all steps of methods shown and/or described with respect to any one or more of FIGS. 1-9, though such is not necessarily required. For example, the snorkel 428 may alternatively be used on its own and/or in other manners. In exemplary embodiments, without limitation, the snorkel 428 may be configured for use with, and/or used with, at least some of the components of the systems 100 and/or 200, and/or steps of the methods related thereto, without limitation. For example, without limitation, the snorkel 428 may be used with vented or unvented spikes 112, 212, such as with or without the channel 126.

In exemplary embodiments, without limitation, the snorkel 428 may be configured for insertion through and/or adjacent to the spike 112, 212. In exemplary embodiments, the container 122, 222 may comprise a puncturable seal located on an otherwise open end thereof. The seal may be of sufficient size to accommodate at least a portion of the spike 112, 212 and the snorkel 428. The seal may comprise a foil, rubberized material, combinations thereof, or the like. The snorkel 428 may be punctured through said seal adjacent to at least the portion of the said spike 112, 212 punctured through the seal. A first end 417 of fluid passageway 462 of the snorkel 428 in such embodiments may be sharpened (e.g., angled) to facilitate such puncture, though a blunted (e.g., smoothed), or other type of end may be utilized. The first end 417 may be open, thereby providing fluid connection between an air pocket within the container 122, 222 and the ambient environment, such as by way of a hollow, interior passageway extending through the snorkel 428. The hollow, interior passageway may extend through, and may include, the fluid passageway 462.

The snorkel 428 may comprise a handle 452. The handle 452 may be configured for a user to grip to manipulate the snorkel 428. The fluid passageway 462 may extend into the handle 452 in exemplary embodiments, without limitation. In this way, access to the hollow, interior passageway may be provided at a second end portion 429 of the snorkel 428, which may be defined, at least in part, by the handle 452.

The fluid passageway 462 may include one or more segments. For example, without limitation, the fluid passageway 462 may include one or more linear, or at least substantially (e.g., within 10%) linear, segments 460, such as along a longitudinal axis thereof, (hereinafter the linear segment(s) 460) and/or one or more non-linear segments 431 (e.g., curved), such as along a longitudinal axis thereof. In exemplary embodiments, without limitation, each of the non-linear segments 431 is connected to one or more of the linear segment(s) 460. Where more than one linear segment 460 is utilized, a longitudinal axis of the linear segments 460A, 460B may extend at an angle relative to one another, such as due to an interposed non-linear segment 431. In exemplary embodiments, without limitation, the fluid passageway 462 comprises a first linear segment 460A, a second linear segment 460B, and a non-linear segment 431 interposed between the first and second linear segments 460A, 460B.

The fluid passageway 462 may, optionally, include one or more shaped segments 454. The shaped segment(s) 454 may be located within the handle 452, though such is not necessarily required. The shaped segment(s) 454 may form a funnel, conical, and/or cylindrical shape, by way of non-limiting example. The shaped segment(s) 454 may form part of the hollow, interior passageway. While a differently shaped segment 454, such as relative to other of the segment(s) 460 are sometimes shown and/or discussed, the shaped segment 454 does not necessarily have to be of a different shape. For example, without limitation, the various fluid passageway 462 segments may be of a same shape, such as generally tubular in shape. The shaped segment(s) 454 may be those located, at least in part, within the handle 452.

The various segments of the fluid passageway 462 may be interconnected. Connection may be made by way of adhesive, welding, bonding, combinations thereof, or the like. Alternatively, or additionally, some or all of the segments of the fluid passageways 462 may be integrally formed.

The various segments of the fluid passageway 462 may be relatively inflexible. For example, without limitation, the various segments of the fluid passageway 462 may be configured to at least substantially (e.g., within 10%) maintain their shape during manual use in the manner described herein.

The fluid passageway 462 may be arranged, in exemplary embodiments without limitation, to permit a second end 458 of the fluid passageway 462 and/or the second end portion 429 of the snorkel 428 to extend a distance from an outer surface of the spike 112, 212 and/or container 122, 222, such as to facilitate access to said second end portion 429 of the snorkel 428 and/or second end 458 of the fluid passageway 462, and thereby the hollow, interior passageway, when said snorkel 428 is inserted into said container 122, 222. This arrangement may, alternatively or additionally, permit ease of handling and/or manipulation, such as by providing an ergonomic angle of use. This may, alternatively or additionally, provide ease of access to the spike 112, 212, container 122, 222, and/or other medical equipment.

The snorkel 428 may, optionally, comprise a cover 464. The cover 464 may be attachable to and/or part of the handle 452, though such is not necessarily required. For example, in other exemplary embodiments, the cover 464 may be attached to the handle 452, such as by adhesive, snap fit, and/or friction fit. Alternatively, without limitation, the cover 464 may be integrally formed with the handle 452.

At least part of at least one of the fluid passageway 462 may extend through the cover 464 to, or at least partially through, the handle 452. In exemplary embodiments, without limitation, some or all of the non-linear segment(s) 431 extends within the cover 464 and at least some of the second linear segment 460B is located within the handle 452. Optionally, at least part of the second linear segment 460B may extend within the cover 464. Regardless, only the first linear segment 460A, or part of the first linear segment 460A, may be otherwise exposed in exemplary embodiments, without limitation. For example, without limitation, the first linear segment 460A may still extend within the protective cover 132, where utilized.

A variety of shapes, sizes, and/or number of non-linear segments 431 may be utilized. A variety of shapes, sizes, and/or number of linear segments 460 may be utilized. While fluid passageway 462 are sometimes shown and/or described, such as having a circular, or substantially (e.g., within 10%) circular, cross section, such as forming a tube or tubular passageway, other size and/or shape cross sections may be utilized and/or the cross section may vary through the snorkel 428 and/or the fluid passageway segment 460. For example, without limitation, the cross section of the fluid passageway 462 may be square, rectangular, triangular, ovoid, some combination thereof, or the like.

One or more fluid control devices 430, 430′ may be provided at the second end portion 429 of the snorkel 428, such as at and/or as part of the handle 452. The fluid control devices 430, 430′ may comprise a valve, filter, combinations thereof, or the like.

In exemplary embodiments, the snorkel 428 comprises a first fluid control device 430 in the form of a cap (hereinafter also “cap 430”). The cap 430 may be hinged or removable. When closed, the cap 430 may provide cover and/or at least a partially (if not fully) seal the second end portion 429 of the snorkel 428. The cap 430 may be connected to the handle 452, such as by way of adhesive, friction fit, snap fit, a mating relationship, combinations thereof, or the like. For example, without limitation, the cap 430 may comprise a base and a cover. The base may be formed as a collar which fits within a ring-shaped depression in handle 452, by way of non-limiting example. The base may, alternatively, be an elastic band which fits to the handle 452, without limitation. Alternatively, the cap 430 may be integrally formed with the handle 452. The cover of the cap 430 may be fully removable from, or preferably but optionally, tethered to the base of the cap 430 such as by a portion of the cap 430 or flexible member (e.g., cable, string, combinations thereof, or the like), thereby facilitating hinging movement of the cover and/or removal of the cover.

The snorkel 428 may, alternatively, or additionally, comprises a second fluid control device 430′ in the form of a filter (hereinafter also “filter 430′”). The filter 430′ may take the form of a mesh and/or screen, by way of non-limiting example. The filter 430′ may be located within or below the cap 430 within and/or as part of the handle 452. The filter 430′ is preferably separately formed and installed by adhesive, friction fit, combinations thereof, or the like. The filter 430′ may rest within the segment 454 located within the handle 452 by way of non-limiting example. In exemplary embodiments, without limitation, the cap 430 is configured to selectively cover the segment 454 while the filter 430′ is located above or within the segment 454 such that removal of the cap 430 exposes the filter 430′ as illustrated with particular regard to FIG. 10C, and removal of the filter 430′ exposes the segment 454 as illustrated with particular regard to FIG. 10D, by way of non-limiting example.

A longitudinal axis of the handle 452 may extend at a non-zero, non-180-degree angle (e.g., between 10-80 degrees, without limitation) relative to a longitudinal axis of at least the first linear segment 460A, which may be a longest (at least longitudinally) one and/or only one of the linear segment(s) 460, thereby providing a comfortable handing orientation of the snorkel 428. A longitudinal axis of the second linear segment 460B may extend at an angle between 10 and 75 degrees from the longitudinal axis of at least the first linear segment 460A, by way of non-limiting example, thereby providing a comfortable handing orientation of the snorkel 428. This may, alternatively or additionally, provide ease of access to the spike 112, 212, container 122, 222, and/or or other medical equipment.

The handle 452 may have a relatively large cross-sectional size compared to the fluid passageway 462, thereby providing comfortable handle and ease of manipulation.

Any of the steps shown or described herein may be performed in any order, may omit one or more steps, may repeat one or more steps, or the like.

Any embodiment of the present invention may include any of the features of the other embodiments of the present invention. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention. It is the intention, therefore, to limit the invention only as indicated by the scope of the claims.

What is claimed is:

1. A snorkel for improving fluid flow during medication delivery procedures, said snorkel comprising:
a fluid passageway forming an interior passageway for ambient air; and
a fluid control device located at the fluid passageway and moveable between a closed position and an opened position to control passage of the ambient air at the interior passageway;
wherein the fluid passageway is sized and shaped to permit extension of a first end of the fluid passageway through a seal of a container and above a fluid line of a substance within the container while the container is oriented for infusion or pooling, the substance remains undrained, and a second end of the fluid passageway as well as the fluid control device remains outside of, and spaced apart from, the container.

2. The snorkel of claim 1 wherein:
the fluid control device comprises a cap;
the cap is located at the second send of the fluid passageway.

3. The snorkel of claim 2 wherein:
the fluid passageway is sized to permit extension of the first end of the fluid passageway through the seal of the container adjacent to a spike.

4. The snorkel of claim 3 wherein:
the fluid passageway comprises a first segment and a second segment, wherein the first and second segments are at least substantially linear along a respective longitudinal axis thereof;
the longitudinal axis of the first segment extends at a non-zero, non-180-degree angle relative to the longitudinal axis of the second segment.

5. The snorkel of claim 4 wherein:
the first segment is directly connected to the second segment; and
the interior passageway is formed, in its entirety, by the first segment and the second segment.

6. The snorkel of claim 4 further comprising:
a protective cover extending over the first segment and the second segment.

7. The snorkel of claim 4 further comprising:
a filter located at the fluid passageway.

8. The snorkel of claim 7 wherein:
the fluid passageway comprises a third segment which is non-linear along a longitudinal axis thereof;
the third segment is interposed between the first segment and the second segment.

9. The snorkel of claim 8 wherein:
the longitudinal axis of the third segment forms a curve.

10. The snorkel of claim 9 wherein:
the first, second, and third segments are integrally formed.

11. The snorkel of claim 10 further comprising:
a handle, wherein cap is provided at the handle, and wherein a first portion of the fluid passageway extends through the handle and a second portion of the fluid passageway extends therefrom, and the filter is provided at a first portion of the fluid passageway.

12. The snorkel of claim 11 wherein:
the cap is configured to selectively fit atop and be removed, at least in part, from a second end of the handle to selectively expose the first portion of the fluid passageway to receive the ambient air.

13. The snorkel of claim 12 wherein:
the filter is located below the cap and interior to the handle.

14. The snorkel of claim 13 wherein:
the first portion of the fluid passageway comprises a shaped segment forming a funnel shape;
the first segment extends from the shaped segment.

15. The snorkel of claim 14 further comprising:
a cover extending from the handle, wherein the third segment extends within the cover, the first segment extends within the handle, and the second segment extends outside of the handle and the cover.

16. The snorkel of claim 15 wherein:
a longitudinal axis of the handle extends at a non-zero, non-180-degree angle relative to the longitudinal axis of the second segment.

17. A delivery system for improving fluid flow during medication delivery procedures, said delivery system comprising:
the snorkel of claim 1;
the container; and
a spike for puncturing the seal adjacent to the snorkel, wherein the snorkel is separate from, and independently movable relative to, the spike.

18. The delivery system of claim 16 further comprising:
an IV drip chamber configured for fluid connection with the spike; and
tubing configured for fluid connection with the IV drip chamber.

19. A delivery system of claim 16 wherein:
the spike comprises a vent.

20. A method for improving fluid flow during medication delivery procedures, said method comprising:
providing the delivery system of claim 16;
puncturing the seal of the container with the spike;
separately puncturing the seal of the container with the first end of the fluid passageway of the snorkel;
moving the snorkel through the seal and relative to the spike such that the first end of the fluid passageway is elevated above the fluid line of the substance within the container while the substance remains undrained, the fluid control device is in the closed position, and the second end of the fluid passageway as well as the fluid control device remains outside of, and spaced apart from, the container and the spike;
orienting the container for infusion or pooling;
opening the fluid control device of the snorkel to permit the ambient air to pass through the interior passageway; and
passively draining the substance through the spike while leaving the fluid control device in the opened position to allow the ambient air to enter the container by way of the interior passageway, and thereby avoiding passing the ambient air though the spike and the substance.
